(19) United States
(12) Patent Application Publication
(10) Pub. No.: US 2003/0125764 A1
(43) Pub. Date: Jul. 3, 2003

(54) Catheter

(76) Inventors: Eamon Brady, Elphin (IE); Brendan Casey, Barna (IE); Anton Clifford, Corrundulla (IE); John Neilan, Gort (IE); Gary Fahey, Rosscahill (IE)

Correspondence Address: Finnegan, Henderson, Farabow,, Garrett & Dunner, L.L.P., 1300 I Street, N.W., Washington, DC, 20005-3315, US

(21) Appl. No.: 10/180,981

(22) Filed: Jun. 27, 2002

Related U.S. Application Data
(60) Provisional application No. 60/301,592, filed on Jun. 29, 2001.

(30) Foreign Application Priority Data
Jun. 27, 2001 (IE) 2001/0592

(51) Int. Cl. A61M 29/00

(52) U.S. Cl. 606/200

(57) ABSTRACT

A catheter 100 for delivery or retrieval of an embolic protection filter 101 has a distal pod 102 to house the filter 101. The filter 101 is deployed by means of an inflation balloon or sac 103 and the catheter has a proximal stop 104 for the sac. A proximal catheter shaft 106 has an inflation lumen for inflating the sac 103 from a delivery retracted configuration to a longitudinally extended configuration to deploy the filter 101. A transition mid section shaft 109 extends proximally from the pod 102 and defines a rapid exchange exit port 105 for a guidewire 107.

### CROSS REFERENCE TO RELATED APPLICATIONS

This application is related to, and claims the benefit of, the following patent applications, namely: Irish Patent Application No. 2001/0592, filed Jun. 27, 2001; and U.S. Patent Application No. 60/301,592, filed Jun. 29, 2001; all of which are hereby incorporated by reference in their entirety.

### FIELD OF THE INVENTION

This invention relates to a catheter suitable for delivery of an embolic protection filter through a vasculature over a rapid exchange guidewire, and deployment and/or retrieval of the filter at a desired site in the vasculature. In particular this invention relates to a catheter which facilitates rapid exchange of the catheter over the guidewire during deployment and/or retrieval of the filter from or to the catheter.

Exchange of a catheter over a guidewire using a rapid exchange arrangement enables an interventional procedure to be performed by a single operator in a fast, efficient manner.

There is a need for a catheter which facilitates both rapid exchange delivery and rapid exchange deployment or retrieval of an embolic protection filter.

### SUMMARY OF THE INVENTION

According to the invention there is provided a catheter comprising:

i. a catheter shaft;

ii. a pod defining a reception space for an embolic protection filter and an energy converter to convert energy into a translational movement of the filter relative to the pod;

iii. the filter being movable relative to the pod to facilitate deployment of a filter from within the reception space or retrieval of a filter into the reception space.

In one case the pod is movable relative to the shaft. In another the pod is fixed relative to the shaft.

In one embodiment the catheter shaft has a guidewire opening located a substantial distance distally of a proximal end of the catheter for rapid exchange of the catheter over a guidewire.

In a preferred embodiment the energy converter is configured to convert potential energy into a translational movement of the filter relative to the pod.

Preferably the energy converter is configured to convert energy in the form of a pressurised fluid into a translational movement of the filter relative to the pod.

In one particularly preferred embodiment the energy converter comprises an expandable chamber into which a pressurised fluid may be passed to move the filter relative to the pod. Most preferably the expandable chamber is located substantially at a distal end of the catheter.

In one embodiment the catheter comprises an expansion lumen in fluid communication with the expandable chamber for passing a pressurised fluid from a proximal end of the catheter to the expandable chamber.

Most preferably the expandable chamber is defined by an inflatable sac. Inflatable sac's provide a number of use advantages. Firstly there are no sliding parts and thus providing a sealed system is relatively easy. The hoop properties of the sac can be controlled so as to minimise frictional forces. This ensures that the expansion occurs only in the desired direction.

In this case preferably the sac comprises an inner membrane and an outer membrane defining the chamber therebetween. Preferably the sac defines a central lumen through which a guidewire may pass.

In one case the central lumen is defined by the inner membrane.

In one embodiment a sac is engageable with a guidewire for gripping a guidewire. In this case preferably the sac comprises an inner membrane, which, on expansion, grips the guidewire.

In one embodiment the sac is of a non compliant material.

In another embodiment the sac is of an elastomeric material.

Preferably the sac has an abutment surface. The abutment surface is preferably defined by a distal end of the sac.

In a preferred embodiment the energy converter is an inflation sac which is movable from a longitudinally retracted configuration to a longitudinally extended configuration.

In one case the sac is concertinered in the retracted configuration.

In another embodiment the sac is of spiral form.

In one arrangement a transition is provided between the catheter shaft and the pod. The transition may comprise a mid section shaft.

Preferably the transition defines a guidewire exit port. Ideally the exit port is a rapid exchange exit port.

The transition may taper proximally from the pod towards the shaft.

The longitudinal axis of the transition may be offset from the axis of the catheter shaft.

In one embodiment the catheter shaft extends through the transition for fluid communication with the expandable chamber.

In one embodiment the catheter has a stop to control movement of the energy converter.

In one case the stop is a proximal stop to limit proximal movement of the energy converter. In another case the stop is a distal stop to limit distal movement of the energy converter.

The stop may be provided on the catheter shaft and/or the pod.

In a preferred embodiment the catheter shaft has only a single lumen. In this case the lumen is preferably an inflation lumen.

Most preferably the catheter shaft is trackable.

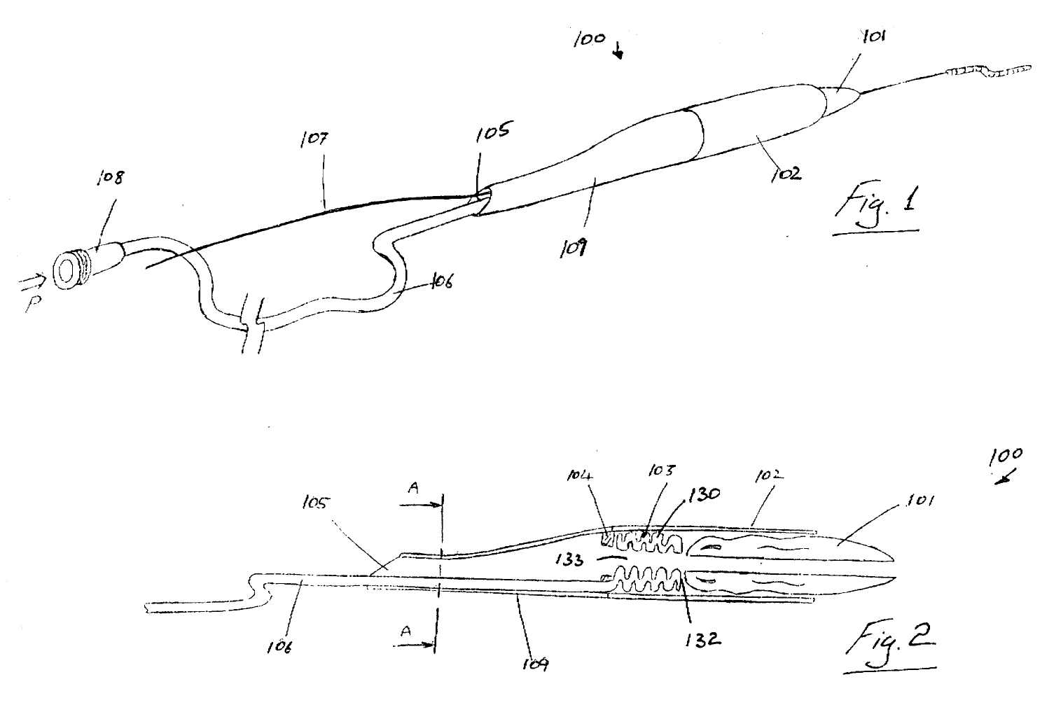

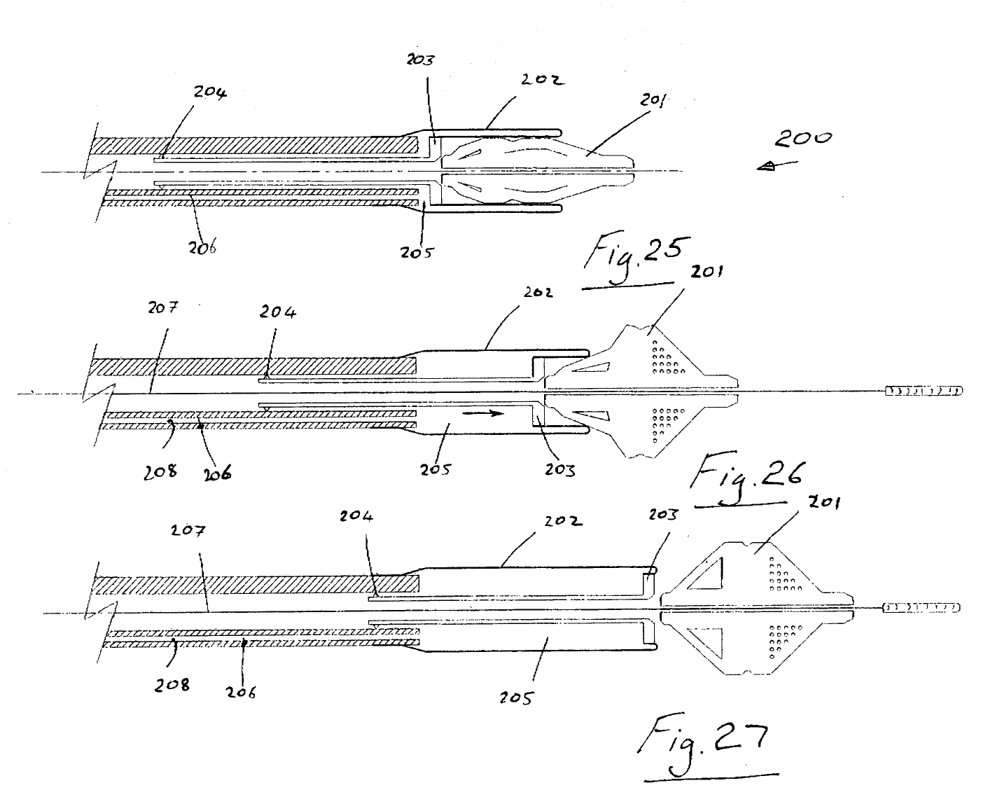

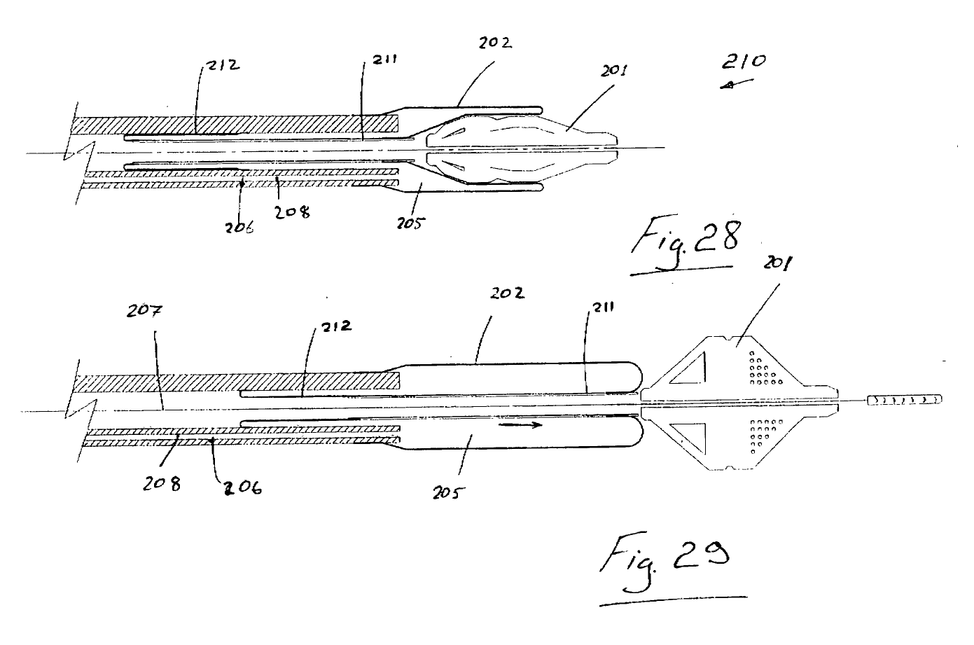

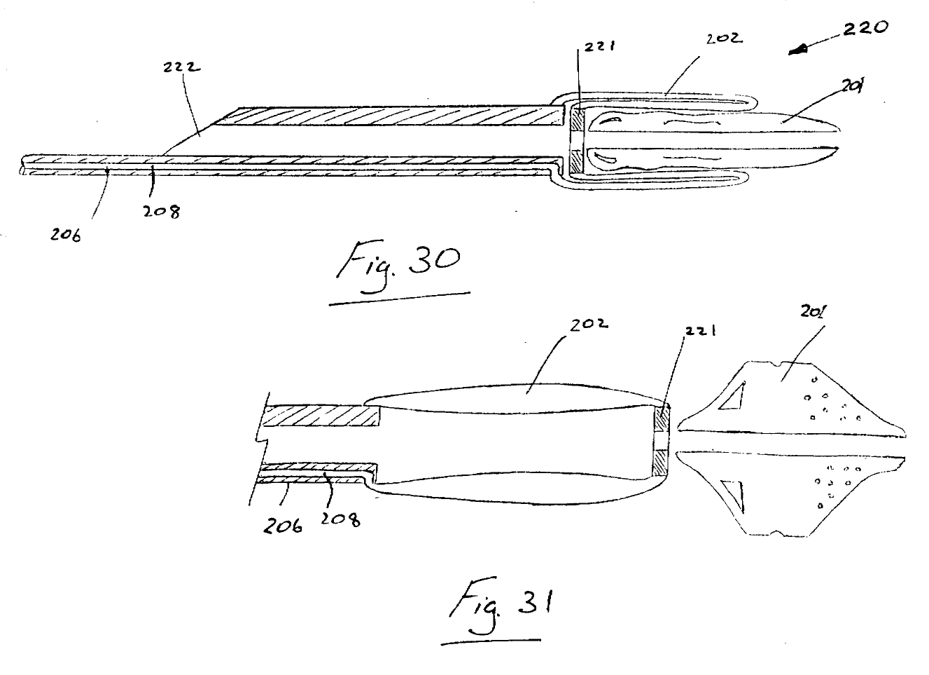

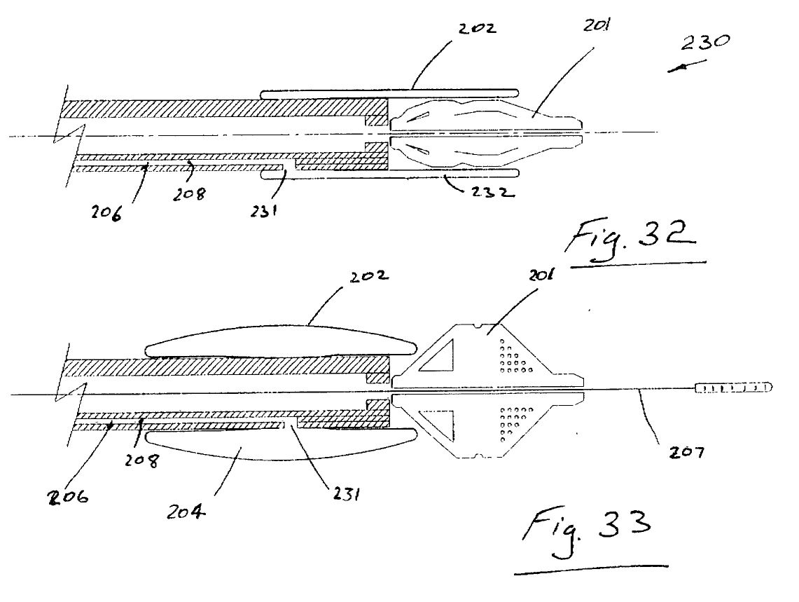

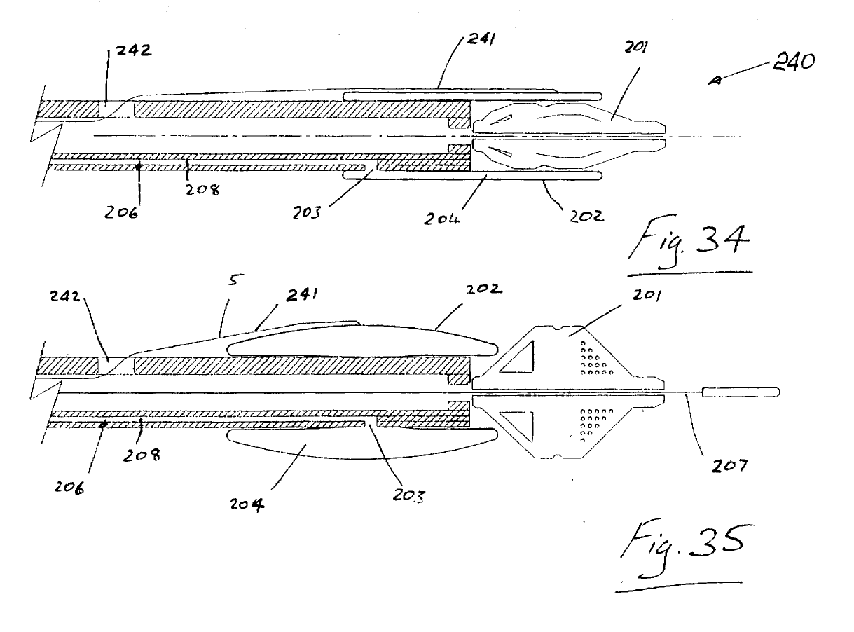

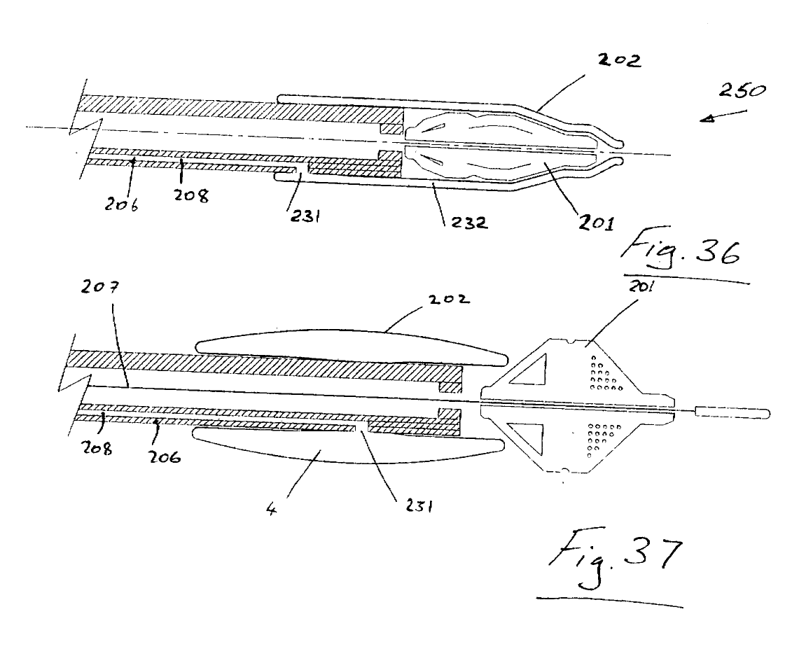

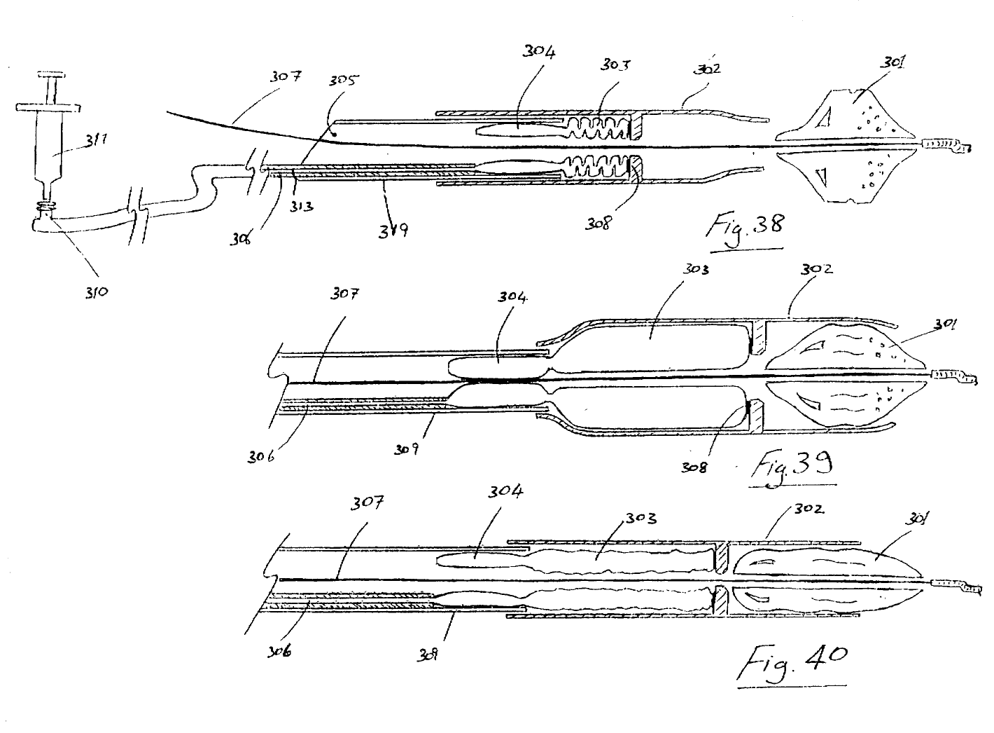

The catheter shaft may have a reinforcement. same reference numerals. In this case a proximal shaft is omitted. The operating procedure is the same as for FIGS. 1 to 6 above. This embodiment shows the distal end of the catheter similar to FIGS. 1 to 6 except with no mid section shaft. The distal end of the catheter consists only of the pod 102 containing the deployment balloon 103 and the loaded filtration element 101. The deployment balloon 103 is shown bonded to the pod at 104 and is shown with a bellows construction to improve folding when collapsed. An inflation syringe 111 is shown in FIG. 9. This embodiment is primarily for a rapid exchange format.

The mid section shaft is removed in this design to improve the trackability of the distal section of the catheter.

Referring to FIGS. 10 and 11 there is illustrated another delivery catheter 120 according to the invention in which parts similar to those of FIGS. 1 to 3 are assigned the same reference numerals.

The operating procedure is similar to the catheter of FIGS. 1 to 3. The catheter 120 is similar to FIGS. 1 to 3 except that a guidewire sleeve 121 has been placed in the lumen of the deployment balloon 103. This sleeve 121 makes it easier to thread the guidewire 107 through the lumen of the balloon 103 in the collapsed configuration when the catheter is threaded onto the guidewire 107. It also reduces friction between the collapsed deployment balloon 103 and the guidewire 107 when the catheter is advanced into a vessel.

When the deployment balloon 103 is inflated with saline solution it expands in the distal direction as described above for FIGS. 1 to 3. As it expands, it pushes the guidewire sleeve 121 in a distal direction. This in turn pushes the filtration element 101 out of the pod 102 and into the vessel. The friction between the deployment balloon 103 and the guidewire sleeve 121 can be minimised by the application of a low friction coating or lubricant to the guidewire sleeve 121.

Ideally the engagement surface is configured to engage a tubular member of a filter. Preferably the tubular member defines a guidewire lumen therethrough.

In another embodiment the catheter comprises a catheter shaft and a pod and the pod defines a guidewire exit port. Preferably the proximal end of the pod defines the exit port. A stop may be provided in the pod. The stop may be a proximal stop. The stop is preferably defined by the pod.

In one embodiment a guidewire sleeve is provided in the pod. Preferably the guidewire sleeve defines a distal abutment.

In another embodiment the pod is of an expansile material.

In one embodiment the inflation lumen for the sac is substantially concentric with the guidewire lumen over at least a portion of the length thereof.

In a preferred embodiment the sac is of a coil form. The coil is preferably of a spiral form.

In one embodiment the catheter has a wire gripper.

Preferably the wire gripper is provided by an expandable sac which, in an expanded configuration, is used to grip a guidewire.

In another embodiment the pod at least partially provides the energy converter.

In a further embodiment the pod is longitudinally movable relative to the catheter shaft. Preferably the pod is movable from a retracted configuration to an extended configuration for deployment of a filter from the pod or for retrieval of a filter into the pod.

In one arrangement the pod is tethered. In this case a tether may extend from the pod to a proximal handle end of the catheter.

Typically the tether is a tether wire.

In one embodiment the catheter is a delivery catheter and the pod defines a reception space for delivery of a filter to a desired site.

In another aspect the catheter is a filter retrieval catheter and the pod defines a reception space for a retrieved filter.

According to another aspect the invention provides a method of delivering a filter comprising the steps of:

a. providing a delivery catheter comprising a catheter shaft, an energy converter and a reception space;

b. placing a filter in the reception space;

c. advancing the delivery catheter and filter through the vasculature to a target deployment site;

d. deploying the filter at the target site with the assistance of fluid pressure.

Preferably the method further comprises the step of removing the delivery catheter.

Preferably the force of delivery is delivered entirely by fluid pressure.

In a preferred embodiment the delivery catheter and filter are advanced through the vasculature over a guidewire. Preferably the delivery catheter and filter are advanced through the vasculature on a guidewire.

The invention also provides a method of retrieving a filter comprising the steps of:

a. providing a retrieval catheter comprising a catheter shaft, an energy converter and a reception space;

b. advancing the retrieval catheter through the vasculature to the retrieval site;

c. retrieving the filter at the target site with the assistance of fluid pressure.

In this case preferably the method further comprises the step of removing the retrieval catheter and filter. Preferably the force of retrieval is delivered entirely by fluid pressure.

In one embodiment the retrieval catheter is advanced through the vasculature over a guidewire.

The invention further provides a method of deploying or retrieving a filter comprising the steps of:

a. displacing a volume of fluid at a proximal end of a catheter;

b. converting the displaced volume of fluid into a displacement at the distal end of a catheter;

c. the displacement effecting a deployment or retrieval of a filter.

The delivery catheter of the invention is particularly suitable for delivering an embolic protection filter through a vasculature over a guidewire, and deploying the embolic protection filter at a desired site in the vasculature. In this case, the distal portion of the catheter body is thin-walled, for example with a wall thickness in the range of from 0.0005″ to 0.00075″. The distal portion is preferably of the material polyethyleneterephthalate (PET), or polytetrafluoroethylene (PTFE).

### BRIEF DESCRIPTION OF THE DRAWINGS

The invention will be more clearly understood from the following description of some embodiments thereof, given by way of example only, with reference to the accompanying drawings, in which:—

a. FIG. 1 is a perspective view of a delivery catheter with a distal filter threaded over a guidewire;

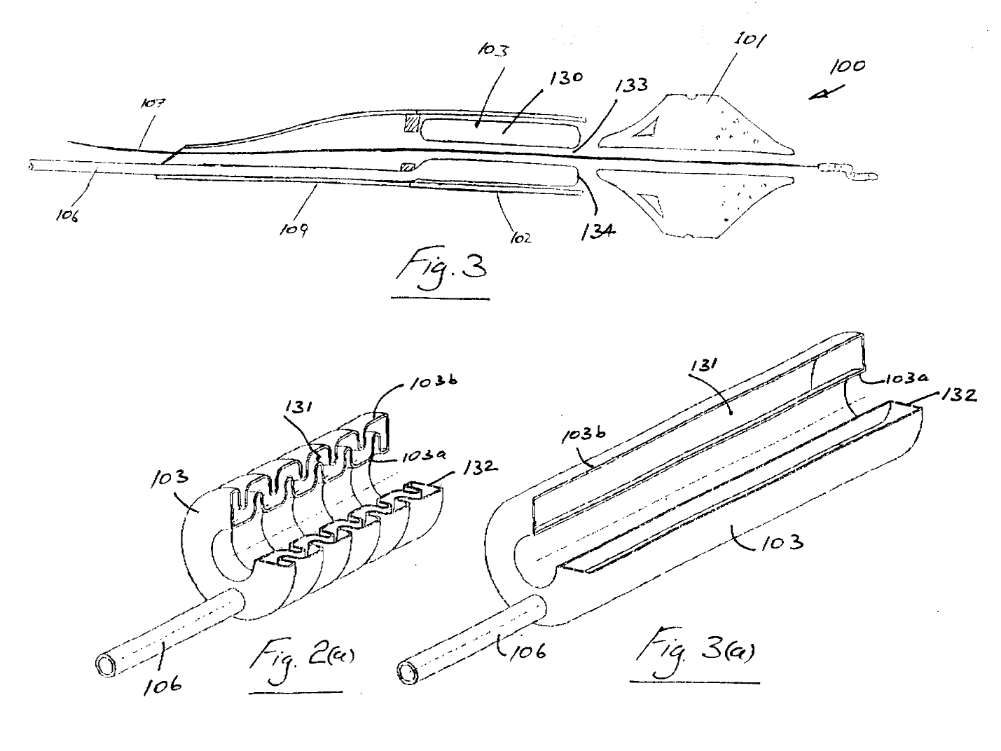

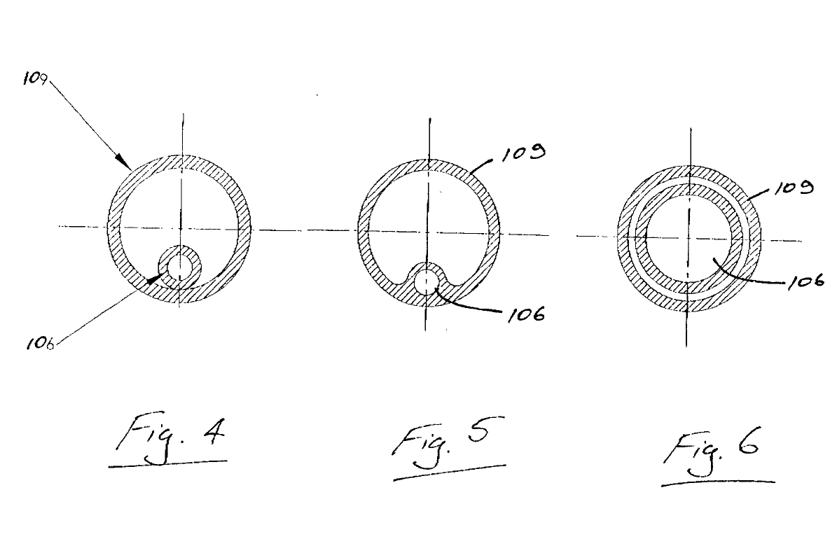

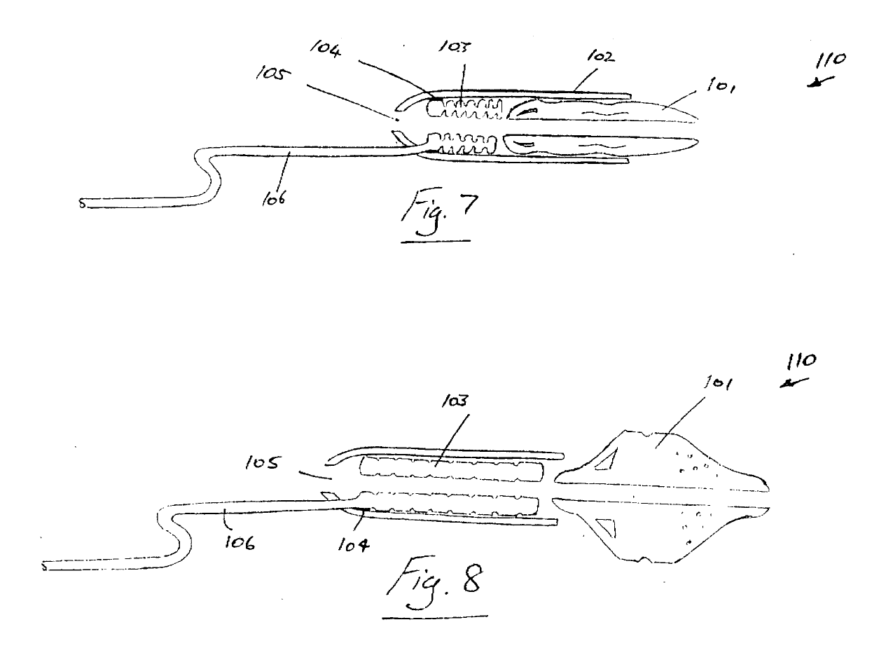

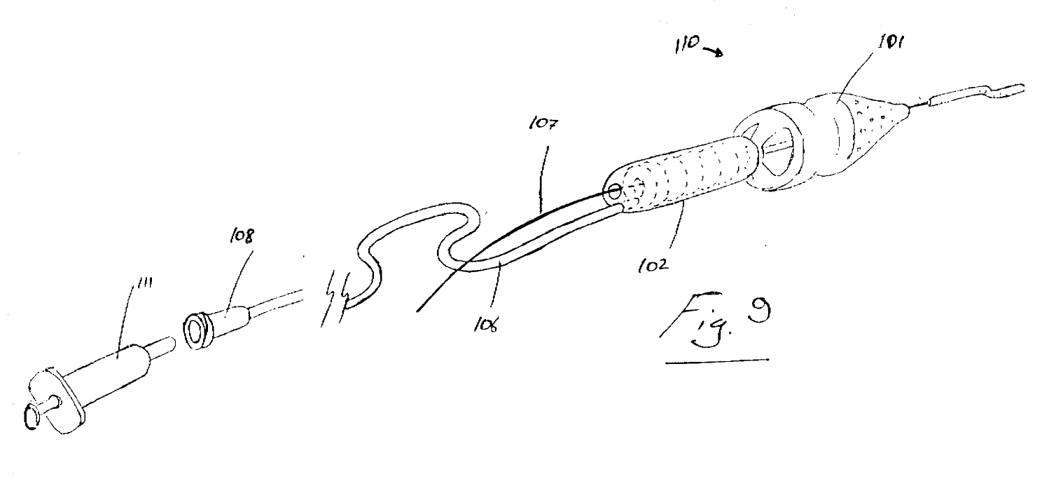

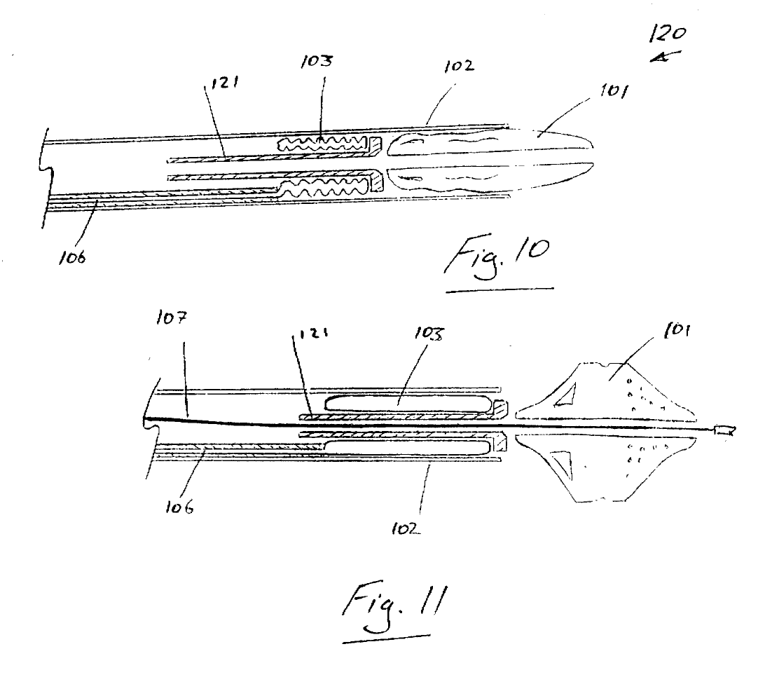

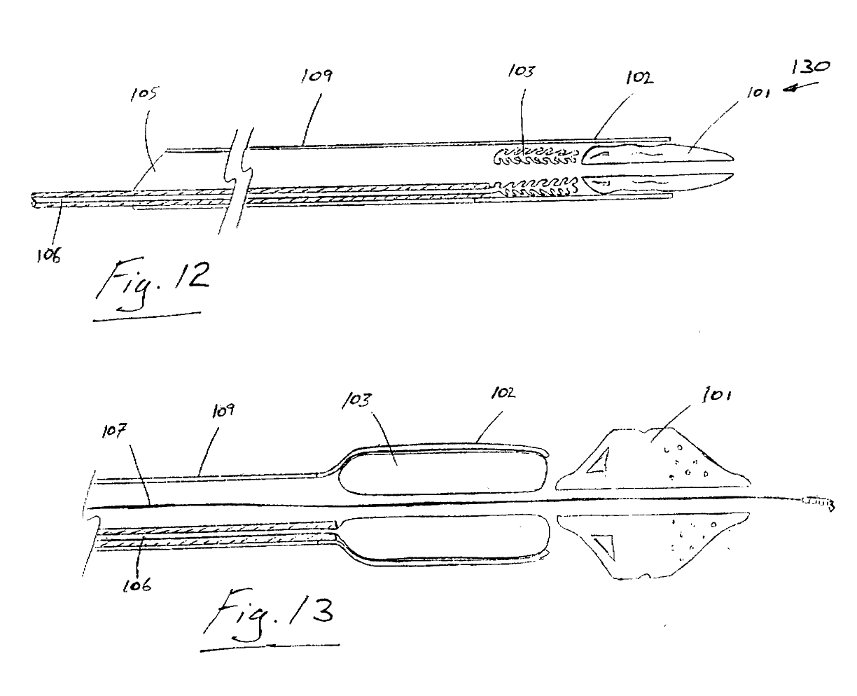

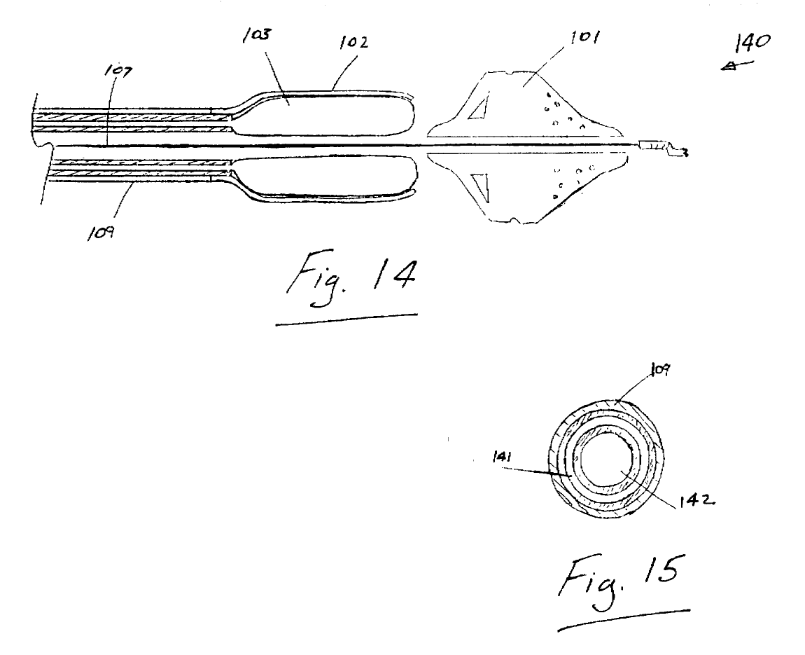

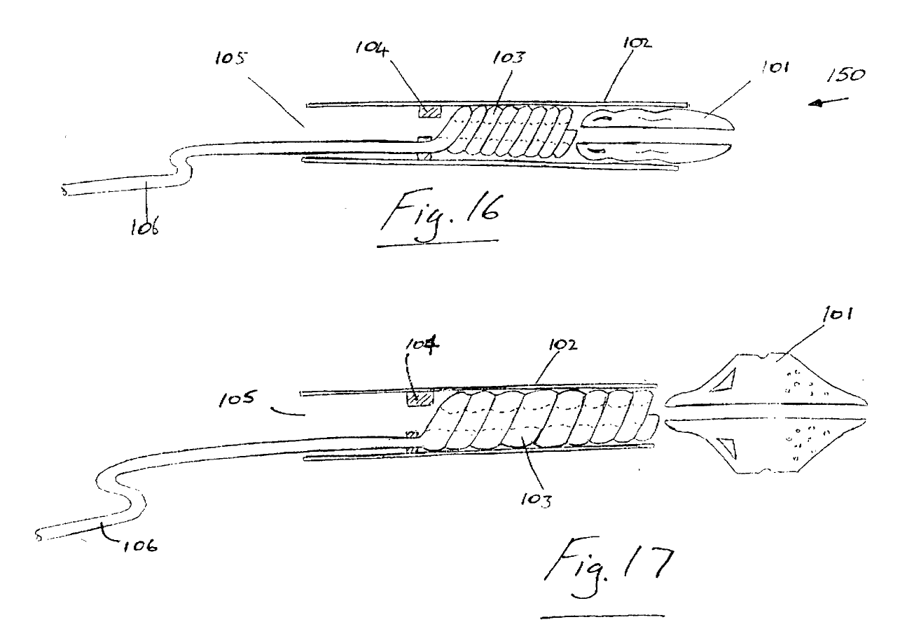

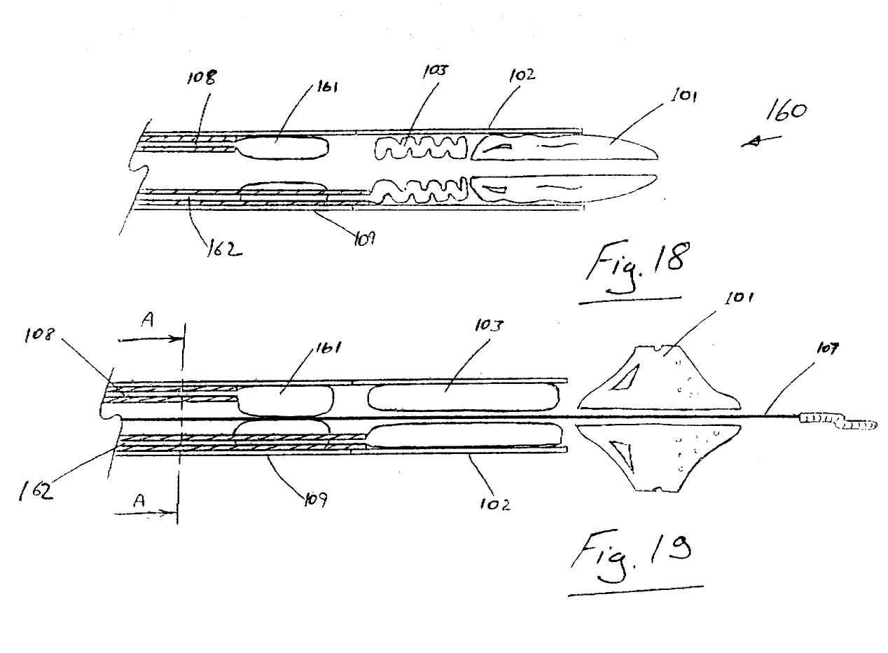

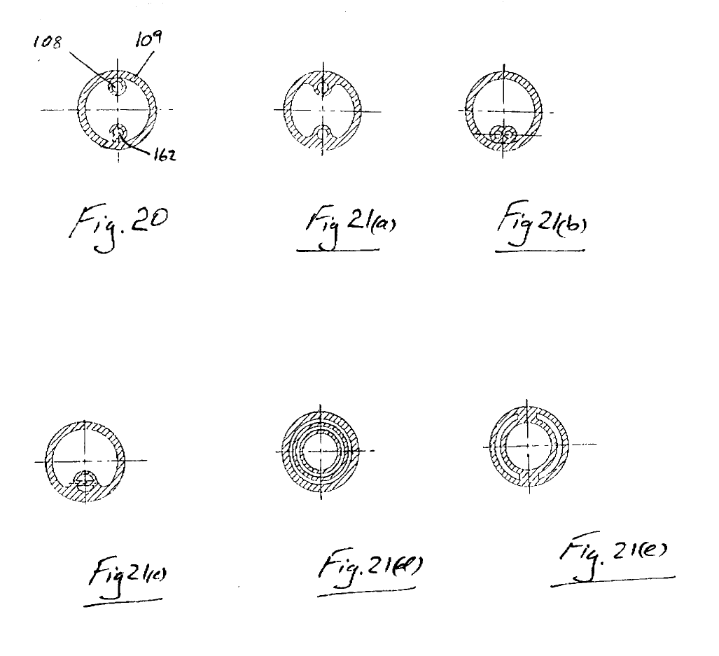

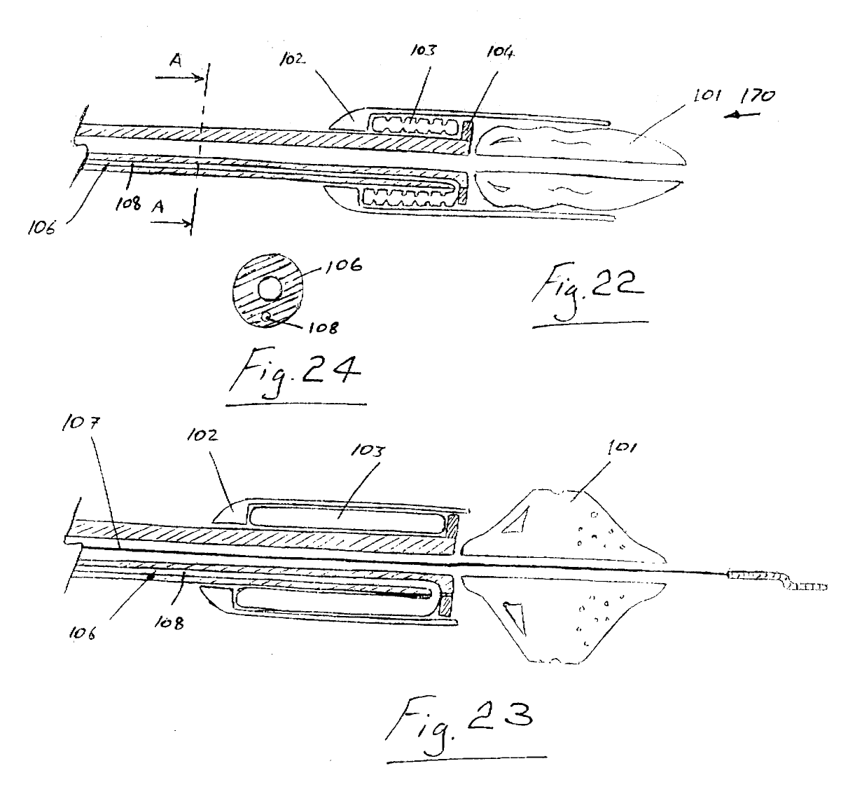

b. FIG. 2 is cross sectional view of the catheter of FIG. 1 with the filter loaded in a pod;

c. FIG. 2a is a perspective, partially cut-away view of an expandable sac in a retracted configuration;

d. FIG. 3 is a cross sectional view of a distal section of the catheter of FIGS. 1 and 2 with a filter deployed in a vessel;

e. FIG. 3a is a perspective, partially cut-away view of the expandable sac in an extended deployed configuration.

f. FIG. 4 is a cross sectional view on the line A-A in FIG. 2;

g. FIG. 5 is a cross sectional view similar to FIG. 4 of an alternative catheter;

h. FIG. 6 is a cross sectional view similar to FIG. 4 of another catheter;

i. FIG. 7 is a cross sectional view of another catheter with a filter loaded into a distal pod of the catheter;

j. FIG. 8 is a cross sectional view of the catheter of FIG. 7 with a filter deployed in a vessel;

k. FIG. 9 is a perspective view of the catheter of FIGS. 7 and 8 with a filter deployed and a guidewire in position.

l. FIG. 10 is a cross sectional view of a distal section of an alternative catheter with a filter loaded into a distal pod of the catheter;

m. FIG. 11 is a cross sectional view of the catheter of FIG. 10 with the filter deployed and a guidewire in position;

n. FIG. 12 is a cross sectional view of a distal section of another catheter with a

o. filter loaded into a distal pod of the catheter;

p. FIG. 13 is a cross sectional view of the catheter of FIG. 12 with the filter deployed and a guidewire m position;

q. FIG. 14 is a cross sectional view of a distal section of another catheter according to the invention with a filter deployed and a guidewire in position;

r. FIG. 15 is a cross sectional view of a shaft of the catheter of FIG. 14;

s. FIG. 16 is a cross sectional view of a distal section of a further catheter with a filter in a distal pod;

t. FIG. 17 is a cross sectional view of the catheter of FIG. 16 with a filter deployed;

u. FIG. 18 is a cross sectional view of a distal section of another catheter with a filter in a distal pod;

v. FIG. 19 is a cross sectional view of the catheter of FIG. 18 with the filter deployed and a guidewire in position;

w. FIG. 20 is a cross sectional view on the line A-A of FIG. 19;

x. FIGS. 21(a) to 21(e) are cross sectional views similar to FIG. 20 of alternative constructions of catheter shaft;

y. FIG. 22 is a cross sectional view of a distal section of another catheter shaft according to the invention with a filter in position in a pod;

z. FIG. 23 is a cross sectional view of the catheter of FIG. 22 with the filter deployed and a guidewire in position;

aa. FIG. 24 is a cross sectional view on the line A-A in FIG. 22;

bb. FIG. 25 is a cross sectional view of a distal section of a catheter shaft with a filter located in a pod;

cc. FIG. 26 is a cross sectional view of the catheter of FIG. 25 with the filter partially deployed;

dd. FIG. 27 is a cross sectional view of the catheter of FIGS. 25 and 26 with the filter fully deployed;

ee. FIG. 28 is a cross sectional view of a distal section of another catheter shaft with a filter located in a pod;

ff. FIG. 29 is a cross sectional view of the catheter of FIG. 28 with the filter deployed;

gg. FIG. 30 is a cross sectional view of a distal section of a catheter shaft with a filter located in a pod;

hh. FIG. 31 is a cross sectional view of the catheter of FIG. 30 with the filter deployed;

ii. FIG. 32 is a cross sectional view of a distal section of a further catheter with a filter loaded into a pod;

jj. FIG. 33 is a cross sectional view of the catheter of FIG. 32 with the filter deployed;

kk. FIG. 34 is a cross sectional view of a distal section of another catheter with a filter in position in a pod;

ll. FIG. 35 is a cross sectional view of the catheter of FIG. 34 with the filter deployed;

mm. FIG. 36 is a cross sectional view of a distal section of another catheter of the invention with a filter in position in a distal pod;

nn. FIG. 37 is a cross sectional view of the catheter of FIG. 36 with the filter deployed;

oo. FIG. 38 is a cross sectional view of a distal portion of a retrieval catheter according to the invention with a filter deployed;

pp. FIG. 39 is a cross sectional view of the catheter of FIG. 38 with the filter partially retrieved;

qq. FIG. 40 is a cross sectional view of the catheter of FIGS. 38 and 39 with the filter fully retrieved;

rr. FIG. 41 is a partially cut-away, perspective view of a delivery catheter according to the invention passing over a guidewire;

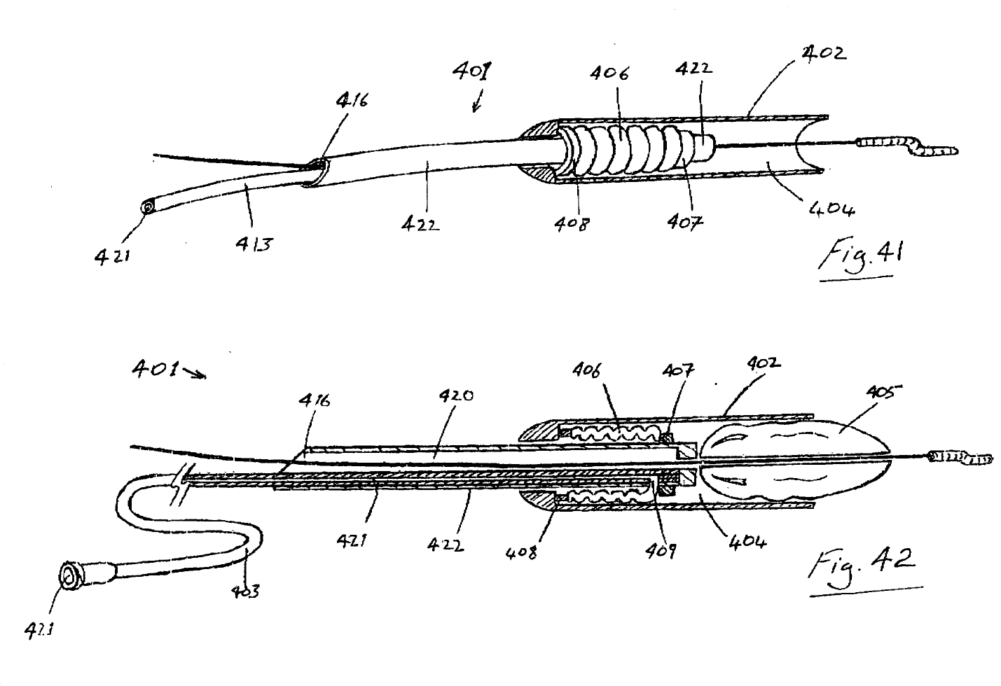

ss. FIG. 42 is a partially cross-sectional, side view of the delivery catheter of FIG. 41 passing over the guidewire;

tt. FIG. 43 is a partially cross-sectional, side view of the delivery catheter of FIG. 41 with the filter deployed;

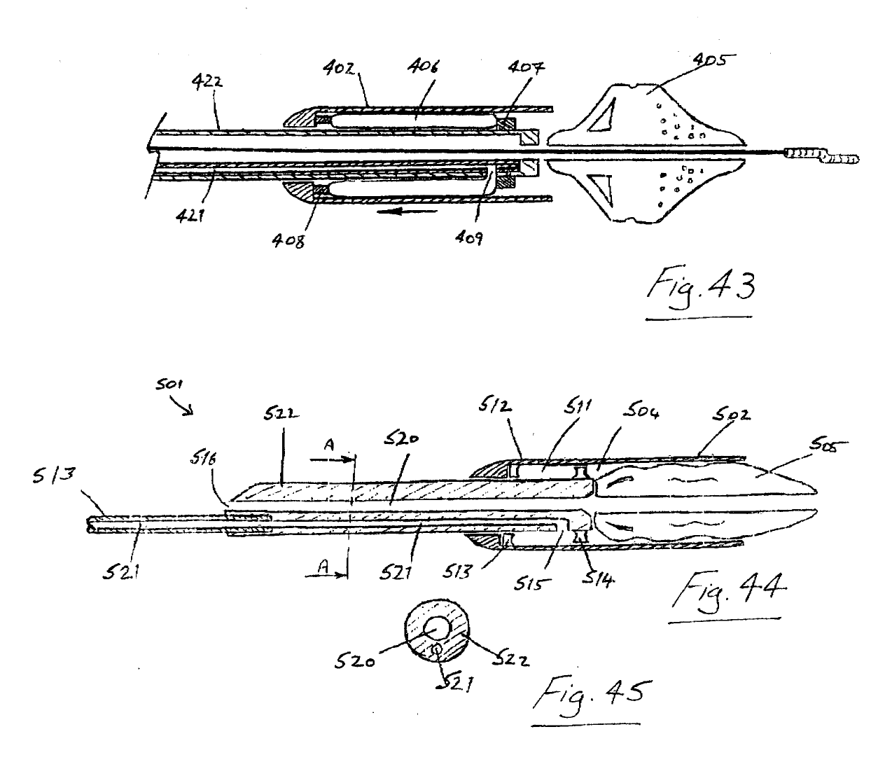

uu. FIG. 44 is a partially cross-sectional, side view of another delivery catheter according to the invention, in use;

vv. FIG. 45 is a cross-sectional view on the line A-A in FIG. 45; and

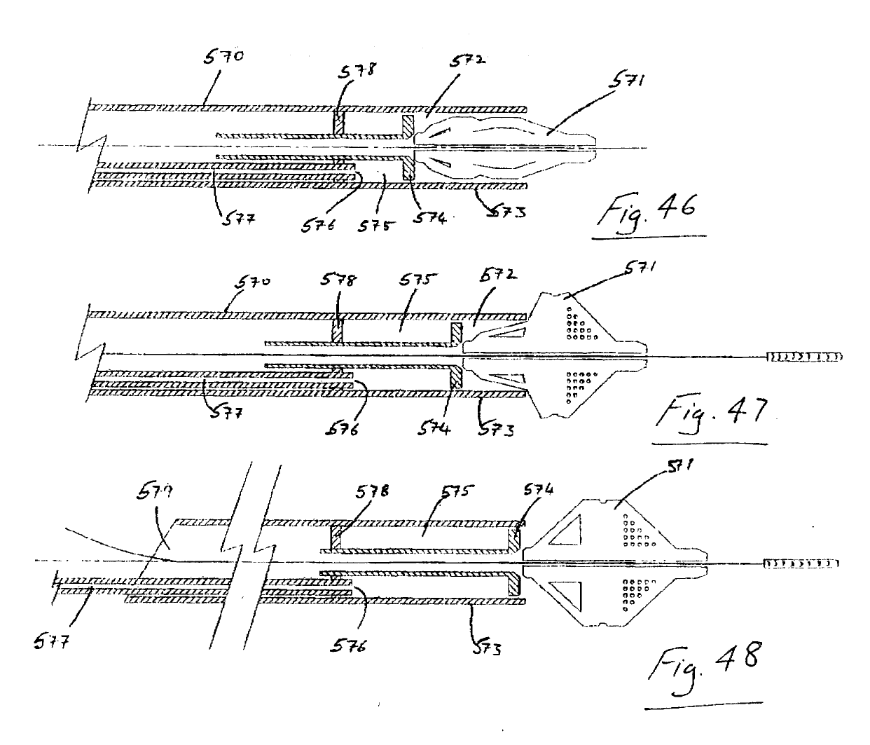

ww. FIGS. 46 to 48 are cross-sectional, side views of another delivery catheter according to the invention, in use.

### DETAILED DESCRIPTION

Referring to the drawings, there is illustrated a catheter according to the invention for delivery or retrieval of an embolic protection filter through a vasculature over a rapid exchange guidewire, and for deployment or retrieval of the filter at a desired site in the vasculature while still enabling the possibility of rapid exchange of the catheter over the guidewire.

The delivery catheter comprises a catheter body having a proximal opening and an opening at the distal end of the catheter body, with a guidewire lumen extending between the openings for passage of a guidewire through the lumen to facilitate rapid exchange of the catheter over the guidewire.

A distal portion of the catheter body defines a reception space for receiving collapsed embolic protection filter during delivery of the filter to a desired site in a vasculature, and the delivery catheter includes means for deploying the filter from the reception space at the desired site in the vasculature. In a retrieval system the catheter has a reception space at a distal end for reception of a retrieval filter.

The catheter is particularly suitable for delivery and deployment of an embolic protection filter, which is received within the reception space but is separate and independent of the delivery catheter, and which is separate and independent of the rapid exchange guidewire. One example of this type of filter is the embolic filter described in International patent application number PCT/IE01/00052, the relevant contents of which are incorporated herein by reference. In a retrieval system such a filter can be retrieved into the catheter.

Referring to FIGS. 1 to 6 there is illustrated a delivery catheter 100 according to the invention in which a filtration element 101 is mounted in a distal catheter pod 102. The filtration element 101 is deployed by means of a balloon or sac 103 and the catheter has a proximal stop 104 for the sac 103. A proximal catheter shaft 106 has an inflation lumen communicating at a distal end with the sac 103 and at the proximal end with a luer fitting 108. The catheter has a mid section shaft 109 with an exit port 105 for a guidewire 107 over which the filter 101 and catheter shaft 109 are tracked.

To prepare the delivery catheter 100, the mid section shaft 109 and catheter pod 102 are flushed to ensure there is no trapped air. The filter 101 is then loaded into the pod 102, for example using a loading funnel and a pusher tool, not shown. Alternatively the filtration element 101 may be pre-loaded into the pod 102.

After prepping, the catheter 100 is advanced over the guidewire 107 to a site at which the filter 101 is to be deployed. The guidewire 107 is threaded through the lumen in the loaded filter 101, then through the central lumen in the deployment balloon 103 until the guidewire 107 exits the catheter 100 at the exit port 105. This allows the catheter 100 to be used as a rapid exchange device. It will be noted that no slot or break in the catheter wall is required for such rapid exchange use. The catheter 100 can also be configured in an exchange length format so that the guidewire 107 exits the catheter 100 at the proximal handle.

The catheter 100 is advanced over the guidewire 107 until it is in the position for filter deployment at a desired site in a vessel. A syringe is then filled with a saline solution and connected to the luer fitting 108 on the proximal end of the shaft 106. Contrast media may be included in the saline solution to improve visibility of the procedure under fluoroscopy. The fluid is injected into the shaft using the syringe to create a hydraulic pressure ‘P’ in the shaft.

The saline solution flows through the lumen in the shaft 106 to the deployment balloon 103. The lumen in the shaft 106 is connected to the deployment balloon 103 so that saline can flow from the shaft 106 into the balloon 103. In this way the hydraulic pressure P is transferred from the proximal end of shaft 106 to the deployment balloon 103.

The deployment balloon 103 is shown in FIGS. 1, 2 and 2a in a collapsed configuration. Inner and outer membranes 103a, 103b of the balloon 103 are folded to shorten the length of the collapsed balloon 103 in the pod 102. An expandable chamber 131 is defined between the inner and outer membranes 103a, 103b. When the hydraulic pressure P acts on the inner and outer membranes 103a, 103b of the balloon 103 it expands in a longitudinal direction. Proximal movement of the balloon 103 is prevented by the stop 104. As the balloon 103 inflates under pressure P it expands in a distal direction and pushes the filter 101 out of the pod 102.

FIGS. 3 and 3a illustrate the deployment balloon 103 in the fully expanded position with the filter element 101 pushed completely out of the pod 102 and deployed in a vessel. After deployment, the hydraulic pressure can be released by disconnecting the syringe and the catheter 100 can be removed from the vessel leaving the deployed filter 101 in position.

The filtration element 101 or other embolic protection device may be of any suitable type as described above. It will be noted that the distal end of the loaded filter provides a smooth transition from the guidewire 106 to the pod 102 when forwarding the device through tortuous anatomy and across lesions.

The distal pod 102 of the catheter contains the deployment balloon 103 and loaded filter 101. It can consist of a thin wall material such as polyethyleneterephthalate (PET) or polytetraflouroethylene (PTFE). It could also consist of an expansile material (see FIGS. 12 to 15) containing polyurethane, silicon or nylon based material such as Pebax. To minimise friction on the inner surface of the pod against the filter and deployment balloon a silicon lubricant or hydrophilic coating may be applied.

The deployment balloon or sac 103 consists of a membrane, which expands in a longitudinal direction when an hydraulic pressure is applied to the inner surface of the membrane. The membrane may be formed from a thin wall material such as polyethyleneterephthalate (PET) folded in the collapsed configuration or may consist of an expansile material such as polytetraflouroethylene (PTFE) or a polyurethane, silicon or nylon based material. A material with different mechanical characteristics in the radial and linear directions such as expanded polytetraflouroethylene (PTFE) could also be used to give high hoop strength with good longitudinal expansion. The deployment balloon 103 contains a central lumen to allow the guidewire to pass through the balloon. A spiral wound tube however could also be used in the device negating the need for a separate central lumen as illustrated in FIGS. 16 and 17. The deployment balloon 103 can be coated in a low friction coating such as a hydrophilic or lubricant to minimise friction.

The proximal stop 104 is fixed to the mid section shaft 109 or pod 102 and prevents the deployment balloon 103 expanding in the proximal direction. This can be achieved by placing a separate stop in the catheter. Alternatively the proximal end of the deployment balloon 103 may be bonded to the mid section shaft, see FIGS. 7 to 9, or the shaft 106 containing the inflation lumen may act as a proximal stop.

The guidewire exit 105 is in this case provided for rapid exchange use. However, unlike conventional rapid exchange systems no slot or break in the catheter wall is required for the guidewire to exit. The position of the guidewire exit 105 is only dependent on the length of the mid section shaft 109.

The catheter shaft 106 contains the inflation lumen and links the deployment balloon 103 with the proximal luer fitting 108. Unlike conventional delivery catheters this device does not require a compressive strength in the shaft to withstand the filter delivery force. By locating the delivery mechanism at the distal end of the catheter 100, no force is exerted on the shaft during deployment. Therefore the compressive strength and stiffness of the shaft can be reduced resulting in a highly trackable shaft. The shaft can consist of a number of material options and composites and can also be reinforced with a single longitudinal wire or multiple wires or with wire braid. FIGS. 4 to 6 show different construction methods for the shaft combined with the mid-section shaft 109. FIG. 4 shows individual mid section and catheter shafts, which may be bonded or connected together. FIG. 5 shows them combined as a multi-lumen tube. FIG. 6 shows a construction where the inflation lumen is concentric with the shaft. The shaft construction shown in FIGS. 4 and 5 can be used in a rapid exchange or exchange length format. The construction shown in FIG. 6 may provide easier assembly of the deployment balloon to the shaft for an exchange length device.

The guidewire 107 illustrated is a conventional guidewire with a stepped increase in diameter at the distal end. This step may provide an abutment to butt against the filter element during retrieval of the filter.

The fitting 108 is a standard luer fitting attached to the proximal end of the shaft. This fitting allows a syringe to be connected to the catheter so that a saline solution can be injected into the device.

The mid section shaft 109 provides a lumen for the guidewire 107 from the pod 101 to the exit port 105. The length of the section 109 can be set so that the guidewire 107 will not be exposed distal to the end of the guide catheter when the device is in use. The mid section shaft 109 can taper down to the guidewire exit 105 or remain parallel for the length of the shaft. Because the inflation lumen in the catheter shaft 106 and the mid section shaft 109 are not concentric, there is no need for the guidewire 107 to break out through the catheter wall for a rapid exchange configuration. The mid section shaft is not essential for the device to operate, see FIGS. 7 to 9, however it protects the guidewire 107 in the vessel. There is minimal compressive or tensile strength requirements of the mid section shaft, therefore it can be designed to be highly trackable.

Because the force of deployment is transmitted by fluid pressure from the user end to the distal end the trackability of the shaft 106 can be improved considerably. Conventional systems require a mechanical element to transmit the deployment force. This element inherently adds stiffness to the shaft construction and makes the shaft less trackable. Furthermore, the cross sectional area of the inflation lumen of shaft 106 is much smaller than the cross sectional area of the inflated balloon 103. Therefore, the tensile forces on the shaft section 106 are small. The fact that the shaft 106 needs to accommodate an inflation lumen only means that the OD of the shaft can be very small. This further improves the trackability by reducing the 2^{nd} moment of area of the shaft. For all of these reasons the shaft 106 of these designs can be made highly trackable.

The only mechanical constraint on this type of design is that the shaft 106 has enough push to cross the lesion to get to the deployment (or retrieval) site. This can be achieved through materials selection or shaft design. A variety of materials could be adopted to this application. In one embodiment the shaft construction is a composite. A preferred composite construction involves the incorporation of high tensile wire in the wall of the shaft. Stainless steel wires are preferred. A variety of configurations are possible including braided systems, spiral wound systems or axial wire in the wall systems.

The inflation of the deployment sac 103 creates a set of local forces. The inflation pressure applies equal and opposite forces to the proximal end of the filter 101 and the proximal stop 104. When these forces exceed the frictional forces between the filter 104 and the wall of the pod 102, the filter 101 will move relative to the pod 102. Because the surfaces of the balloon 103 will move relative to the pod 102 during inflation it is preferred that a low friction coating be applied to these surfaces to minimise frictional losses. The distal end of the inflation sac 103 will have an abutment surface 132. Preferably the abutment surface 132 is square to ensure that the pressure forces are delivered in an axial fashion.

In one embodiment the inflation sac 103 is noncompliant. In this embodiment the inflation sac 103 applies only a small force to the pod 102 and most of the force is delivered to the deployment action. In another embodiment the inflation sac 103 is elastomeric. This system has the advantage that very little folding of the sac 103 is required in its collapsed configuration.

An especially important feature of this design is that a guidewire path is maintained through the centre of the sac 103. This is achieved by providing a central lumen 133. This allows the filter 101 to be loaded into the pod 102 as part of the preparation operation. The prepared device may then be introduced onto a guidewire 107 which has been preplaced across the lesion. This feature is especially advantageous.

Another advantageous feature is that the inflation sac 103 may grip the guidewire 107 during the inflation step. This allows the user to control the relative movement during the deployment step. This wire gripping feature can be controlled by varying the wall thickness of the membrane so as to achieve a better clamping force on the wire 107 at an early stage in the inflation step.

The design of the guidewire exit port 105 is simplified by this invention since there is little need for mechanical force transmission across the exit port 105.

Referring to FIGS. 7 to 9 there is illustrated another delivery catheter 101 which is similar to the catheter of FIGS. 1 to 6 and like parts are assigned the

The sleeve element 121 may be bonded to the distal end of the inflation sac 103. The sleeve element 121 can be provided by a simple tube.

Referring to FIGS. 12 and 13 there is illustrated another deployment catheter 130. The operating procedure is similar to that of the catheter of FIGS. 1 to 3. In this case the catheter pod 102 is made from an expansile material and increases in diameter as the deployment balloon 103 is inflated. Therefore, as the inflation pressure P increases, the outer diameter of the pod 102 increases and the deployment pressure acting on the filter 101 increases until the filter is pushed out of the pod 102 and deployed in a vessel.

Two advantages of this design are that as the diameter of the pod 102 increases, the deployment force required to push the filter 101 out of the pod is reduced. In addition the force acting on the filter as a result of a fixed hydraulic pressure P in the deployment balloon increases significantly for an increase in the diameter of the balloon. The deployment force is proportional to the balloon diameter squared, for a fixed hydraulic pressure.

Referring to FIGS. 14 and 15 the catheter 140 of this embodiment has a shaft construction with a guidewire lumen 142 and a concentric inflation lumen 141. This construction could improve ease of assembly of the deployment balloon to the shaft for an exchange length device.

Referring to FIGS. 16 and 17 there is illustrated another delivery catheter 150 according to the invention. The construction and operating procedure is similar to the catheter of FIGS. 1 to 3 and like parts are assigned the same reference numerals. This catheter 150 in which the deployment balloon 103 is constructed from a tube wound in a spiral and placed inside the pod 102. The deployment balloon 103 could then be formed using the catheter shaft 106. When the filter 101 is loaded in the pod 102 the tubing is collapsed and flattened laterally. When the hydraulic pressure P is applied to the shaft 106 the spiral tubing 103 recovers its shape expanding in the distal direction. The expansion of multiple coils of the tubing gives sufficient expansion to push the filter 101 out of the pod 102. The coiling of the tubing automatically creates a central lumen for the guidewire to pass through when threading the catheter over the wire.

Referring to FIGS. 18 to 20 there is illustrated another delivery catheter 160 which is similar in construction to the catheter of FIGS. 1 to 3 and like parts are assigned the same reference numerals.

There is a significant difference in the operating procedure for this catheter 160 because there is an additional step required in the deployment of the filter 101. The device is prepped and forwarded over the guidewire to the site of the filter deployment in the same way as described previously.

This catheter has a wire gripping balloon 161, an associated inflation lumen 162 and luer fitting at the proximal end of the shaft. When the catheter is in position saline solution is injected into the inflation lumen 162 for the wire grip balloon 161. The hydraulic pressure causes the wire grip balloon 161 to expand in a radial direction. When fully inflated this balloon exerts a pressure onto the guidewire 107. This prevents any movement of the catheter relative to the guidewire 107 due to friction between the guidewire and the inflated wire grip balloon 161. The wire grip balloon 161 is prevented from expanding radially outwards by the mid section shaft and is constructed so it does not expand laterally. Materials for this balloon 161 would preferably have a high coefficient of friction such as a low hardness polyurethane, silicon or nylon based material such as Pebax.

The deployment balloon 103 can then be inflated in a similar operation to that of FIGS. 1 to 3 above, causing the filter 101 to be deployed in a vessel. After the filter 101 is fully deployed the hydraulic pressure in the wire grip balloon 161 can be released allowing the catheter to be removed leaving the deployed filter 101 in position.

The benefit of this system is that the catheter 160 is locked in position relative to the guidewire 107 during filter deployment. This can enable greater accuracy in the positioning of the filter 101 in the vessel. Also, by locking the catheter 160 to the guidewire 107 the tensile strength of the guidewire 107 is used to support the catheter.

FIGS. 20 and 21(a) to 21(e) show a number of options for the shaft construction. The options of FIGS. 20 and 21(b) show multiple tubing options. The options of FIGS. 21(a) and 21(c) are multi-lumen versions of the shaft. The options of FIGS. 21(d) and 21(e) show concentric lumens, which are more suited to an exchange length device.

Referring to FIGS. 22 to 24 there is illustrated another catheter 170 according to the invention in which parts similar to those of FIGS. 1 to 3 are assigned the same reference numerals. The prepping and operating procedure for this device is the same as for FIGS. 1 to 3 above. However in this embodiment the pod 102 moves in proximal direction allowing the static filter 101 to expand and deploy in the vessel, rather than filter being pushed out of the static pod in a distal direction.

The deployment balloon 103 is inflated with a hydraulic pressure the same as before, but in this construction the balloon 103 is constrained in the distal direction by a distal stop 171 connected to the catheter shaft. The deployment force acts on the sliding pod 102 moving it in a proximal direction. As the pod 102 moves in the proximal direction it exposes the loaded filter 101 allowing it to expand and deploy in the vessel. Because the filter 101 is static during the deployment procedure it can be positioned very accurately in the vessel. In addition there is no contact between the deployment balloon 103 and the guidewire 107 so there is minimal friction when advancing the catheter 170 over the guidewire 107.

Referring to FIGS. 25 to 27 there is illustrated another construction of delivery catheter 200 in which a filtration element 201 is mounted in a rolling distal pod 202. The filtration element 201 is deployed by means of a piston 203 with a sliding seal 204 defining a fluid cavity 205. A catheter shaft 206 has an inflation lumen 208 for operating the piston 203. The catheter 200 and filter 201 are tracked over a guidewire 207.

The prepping and operating procedure for this device is the same as for FIGS. 1 to 3 above. However in this catheter design the deployment process is different. The pod 202 on this device consists of a flexible membrane, which is rolled back on itself and connected to a piston 203. The saline solution injected into the luer fitting on the proximal end of the shaft creates a hydraulic pressure in the inflation lumen 208 and hence the fluid cavity 205. This pressure exerts a force on the piston 203 causing it to move in a distal direction. As the piston moves distally, the pod 202 unrolls exposing the filter and allowing it to expand and deploy in the vessel.

During deployment the saline solution in the fluid cavity 205 exerts a pressure on the inner and outer membrane of the pod. This separates the two membranes and effectively deletes the frictional component of the deployment force.

The sliding seal 204 minimises leakage of the hydraulic pressure in the fluid cavity 205 as the piston moves laterally. This seal can be formed in a number of ways such as a sliding seal or a membrane seal, see FIGS. 28 and 29.

When the filter is loaded in the catheter a vacuum or negative pressure can be applied to the luer fitting on the shaft. This creates a negative pressure in the inflation lumen 208 and also the fluid cavity 205. This causes the pod 202 to collapse and compress the loaded filter 201, reducing the profile of the catheter 200.

Referring to FIGS. 28 and 29 there is illustrated another catheter 210 which is similar to the catheter of FIGS. 25 and 27 and like parts are assigned the same reference numerals. In this case the catheter has a pusher 211 and a pusher membrane seal 212. The catheter 210 shown here is similar to that shown in FIGS. 25 to 27 except that the piston has been replaced with a pusher 211 constructed from a tube. The sliding seal has also been replaced with a rolling membrane seal 212. This embodiment can improve the ease of assembly and reduce the deployment force by eliminating the friction due to the sliding seal.

Referring to FIGS. 30 and 31 there is illustrated another catheter 220 which is similar to the catheter of FIGS. 25 to 27 and like parts are assigned the same reference numerals. The catheter 220 has a pusher 221 and a guidewire exit 222 is provided for a guidewire 207. The prepping and operating procedure for this device is the same as for FIGS. 1 to 3 above. However in this embodiment the pod 202 consists of a flexible balloon, which is rolled back on itself and connected to a pusher 221. The saline solution injected into the luer fitting on the proximal end of the shaft creates a hydraulic pressure in the inflation lumen 206 and hence in the balloon pod 202. This pressure exerts a force on the inner and outer membranes of the pod 202, which generates stresses in the walls of the pod. To equalise the stresses the pod 202 will try to straighten causing the inner part of the pod 202 to roll forward. As the inner part of the pod 202 is attached to the pusher 221 this also moves forward applying a deployment force to the filter 201. This distal movement of the filter 201 continues until the pod 202 is fully straightened, FIG. 31, and the filter 201 is deployed in the vessel.

The advantage of this design is that no moving seals are required so it is easier to manufacture and assemble. A low friction coating or lubricant could be applied to the balloon pod 202 to minimise the friction when rolling forward.

Referring to FIGS. 32 and 33 there is illustrated another catheter 230 which is similar to the catheter of FIGS. 25 to 27 and like parts are assigned the same reference numerals. In this case the catheter 230 has a balloon cavity 232 with an inflation port 231. The prepping and operating procedure for this device is the same as for FIGS. 1 to 3 above. The rolling pod 202 on this device consists of a flexible membrane balloon, which contains the filter element 201 in the loaded configuration. When the device is in position to deploy the filter 201, the saline solution is injected into the luer fitting on the proximal end of the shaft, as before. This creates a hydraulic pressure in the inflation lumen 206 and hence the balloon cavity 232 via the inflation point 231. This pressure exerts a force on the membrane wall of the rolling pod 202 causing it to expand. This expansion creates hoop stress within the wall of the pod. This stress varies along the length of the pod 202, particularly where there are frictional effects in the area of the pod in contact with the shaft wall. For the pod 202 to reach a state of equilibrium, it must roll in a proximal direction so that stresses in the wall equalise. This proximal rolling of the pod 202 exposes the filter 201 allowing it to expand and deploy in the vessel.

This design of catheter minimises the deployment force because there are no frictional forces to overcome, as the pod 202 rolls off the filter element 201 during deployment. In addition, because the filter 201 is static during the deployment procedure it can be positioned very accurately in the vessel.

When the filter 201 is loaded in the catheter a vacuum or negative pressure can be applied to the luer fitting on the shaft. This creates a negative pressure in the inflation lumen 206 and also the balloon cavity 232. This causes the pod 202 to collapse and compress the loaded filter, reducing the profile of the catheter.

The rolling pod 202 can be pre-stressed or formed from an elastic material to improve the ability of the pod to roll when a hydraulic pressure is applied. The pre-stressed or elastic material configuration can be held in position by friction between the pod membranes when there is low pressure or negative pressure in the pod.

This catheter may be used to occlude the vessel during the deployment of the filter or other device in the vasculature.

Referring to FIGS. 34 and 35 there is illustrated another catheter 240 according to the invention which is similar to the catheter of FIGS. 32 and 33 and like parts are assigned the same reference numerals. In this case there is a tether wire 241 which is threaded through a tether wire exit 242. The prepping and operating procedure for this device 240 is the same as for FIGS. 1 to 3 above. During the deployment procedure the rolling pod 202 performs in the same way as described above in FIGS. 32 and 33. However in this embodiment a tether wire 241 has been connected to the rolling pod 202. This tether wire 241 links the rolling pod 202 with a handle at the proximal end of the catheter. During deployment a pull force is applied to the tether wire 241 to aid the rolling motion of the pod 202.

The tether wire 241 can run through the guidewire lumen of the shaft, exiting the shaft at an exit 242 as shown. Alternatively the tether wire can run through a dedicated lumen in the shaft or in the inflation lumen (6). A tether tube can also be used to replace a wire and may run internally or externally of the catheter shaft.

Referring to FIGS. 36 and 37 there is illustrated another catheter 250 which is similar to the catheter of FIGS. 32 and 33 and like parts are assigned the same reference numerals. The prepping and operating procedure for this device is the same as for FIGS. 1 to 3 above, and the rolling pod 202 deployment action is as described above in FIGS. 36 and 37. In this embodiment the rolling pod 202 completely envelops the loaded filter 201 when the filter is in the loaded configuration in the catheter. By having the distal end of the rolling pod 202 in close proximity to the guidewire a smooth transition from the guidewire to the catheter is provided by the outer membrane of the pod 202. There is no requirement for the distal end of the filtration element to provide a smooth transition.

Referring to FIGS. 38 to 40 there is illustrated a retrieval catheter 300 according to the invention for retrieving a filtration element 301 into an expansile pod 302. The catheter comprises a pusher balloon 303 and a wire grip balloon 304. A catheter shaft 306 has an inflation lumen 313 through which an inflation force is delivered by a syringe 310 connected to a proximal luer fitting 309 of the shaft 306. A guide wire 307 exits through an exit port 305 of the catheter 300. A bond 308 is provided between the balloon 303 and the pod 305.

To prepare the retrieval catheter 300, the mid section shaft 309 and expansile pod 302 are flushed with saline solution to ensure there is no trapped air. After prepping, the device is advanced over the guidewire 307 to the site of the filter deployment. To do this the guidewire 307 is threaded through the central lumen in the pusher balloon 303 and wire grip balloon 304 until the guidewire 307 exits the catheter at the exit port 305. This allows the catheter to be used as a rapid exchange device. It will be noted that no slot or break in the catheter wall is required for rapid exchange use. The device 300 can also be configured in an exchange length format so that the guidewire 307 exits the device at the proximal handle.

The catheter 300 is advanced over the guidewire 307 until it is in the position for filter retrieval with the filter 301 restrained between the retrieval catheter 300 and the distal step 312 on the guidewire 307. A syringe 311 is then filled with a saline solution and connected to the luer fitting 310 on the proximal end of the shaft 306. Contrast media may be included in the saline solution to improve visibility of the procedure under fluoroscopy. The fluid is injected into the shaft using the syringe to create a hydraulic pressure ‘P’ in the shaft.

The saline solution flows through the lumen in the shaft 306 to the wire grip balloon 304 and pusher balloon 303. The lumen in the shaft 306 is connected to the wire grip balloon 304 so that saline can flow from the shaft into the balloon. In this way the hydraulic pressure P is transferred from the proximal end of shaft 306 to the wire grip 304 and pusher balloon 303.

As the hydraulic pressure P increases the wire grip balloon 304 expands in a radial direction. This causes the balloon wall to contact the guidewire 307. Friction between the wire grip balloon 304 and the guidewire 307 prevents any lateral movement of the distal end of the catheter relative to the guidewire 307

Simultaneously the pusher balloon 303 inflates due to the hydraulic pressure, exerting a radial force to the expansile pod 302 and also a lateral force. Movement in the proximal direction is prevented by the wire grip balloon 304, therefore when the lateral force exerted by the pusher balloon exceeds the retrieval force the pusher balloon 303 moves the expansile pod 302 in a distal direction. This pushes the expansile pod 302 over the deployed filter 301 enclosing it within the pod 302.

FIG. 39 shows the pusher balloon 303 in the fully inflated position with the pod 302 over the filter element 301. The wire grip balloon 304 is also fully inflated gripping the guidewire 307. A flow restrictor can be placed in the lumen between the wire grip 304 and pusher balloon 303 to ensure the wire grip balloon 304 is inflated before the pusher balloon 303 inflates. Alternatively the two balloons 303, 304 can have separate inflation lumens in a multi-lumen shaft as illustrated above in FIG. 21.

When the filter 301 is fully enclosed in the pod 302 the hydraulic pressure in the pusher balloon 303 can be released allowing the pod 302 to collapse around the filter 301. This method of retrieval minimises the risk of emboli extrusion as the pod 302 is expanded when forwarded over the filter 301.

When both balloons 303, 304 are deflated, the catheter 300 containing the retrieved filter 301 can be removed leaving the guidewire 307 in position. A bond 308 between the pusher balloon 303 and the expansile pod 302 ensures the pod 302 is removed with the catheter. Alternatively, intersecting steps could be positioned on the proximal end of the expansile pod and the distal end of the midsection shaft.

Allowing the expansile pod 302 to expand radially reduces the hydraulic pressure required to retrieve the filter due to the increased area the pressure is acting on. It also reduces the retrieval force by increasing the diameter of the pod 302.

The wire grip balloon 304 is prevented from expanding radially outwards by the mid section shaft 309 and is constructed so it does not expand laterally. Materials for this balloon would preferably have a high coefficient of friction such as a low hardness polyurethane, silicon or nylon based material such as Pebax.

By gripping the guidewire 307 at the distal end of the catheter, minimal tensile strength is required in the catheter shaft therefore the catheter can be made highly trackable.

Referring to FIGS. 41 to 43, there is illustrated a delivery catheter 401 according to the invention. The distal portion 402 of the catheter body is movable proximally relative to a mid section shaft 422 and proximal shaft 403 of the catheter body to facilitate deployment of an embolic protection filter from a position within the reception space 404. In this case, the embolic protection filter 405 is movable between a collapsed configuration and an expanded configuration. During delivery of the filter 405 to a desired site in a vasculature, the filter 405 remains in the collapsed configuration in the reception space 404 (FIG. 42). Upon deployment of the filter 405 at the desired site in the vasculature, the filter 405 moves to the expanded configuration (FIG. 43).

In this case, the means for deploying the filter 405 is provided by an expandable balloon 406 connected to the distal portion 402 of the catheter body. A distal end 407 of the balloon 406 is fixed to the mid section shaft 422 of the catheter body, and a proximal end 408 of the balloon 406 is fixed to the distal portion 402 of the catheter body, as illustrated in FIG. 42.

The balloon 406 has a fluid port 409 through which an expansion fluid such as compressed air or saline solution may be passed to expand the balloon 406. The catheter body is, in this case, a single lumen tube, with an expansion lumen 421 in communication with the fluid port 409 for passing the expansion fluid into the balloon 406. The guidewire lumen 420 is located within the mid section shaft 422, as illustrated in FIGS. 41 to 43.

Upon expansion of the balloon 406, the internal fluid pressure acting on the walls of the balloon 406 acts to push the proximal end 408 of the balloon 406 proximally relative to the distal end 407 of the balloon 406, and thus move the distal portion 402 of the catheter body proximally relative to the mid section shaft 422 of the catheter body.

The ends 407, 408 of the balloon 406 are completely sealed. Thus during expansion of the balloon 406, and movement of the distal portion 402 of the catheter body proximally relative to the main portion 403 of the catheter body, there is no possibility of leakage of the expansion fluid from the balloon 406. In this way, a wide range of fluids may be chosen for the expansion fluid without the risk of leakage.

As the distal portion 402 moves proximally relative to the mid section shaft 422 of the catheter body, a distal end of the mid section shaft 422 abuts the collapsed filter 405 (FIG. 42) to prevent the filter 405 from moving proximally with the distal portion 402 of the catheter body. In this manner, the filter 405 is uncovered by the proximal movement of the distal portion 402 of the catheter body relative to the mid shaft section 422 of the catheter body, and thus expansion of the filter 405 to the deployed configuration is facilitated (FIG. 43).

The distal portion 402 of the catheter body moves proximally relative to the main portion 403 of the catheter body a distance sufficient to deploy the filter 405, but without interfering with the passage of a guidewire through the rapid exchange guidewire port 416.

The balloon inflatable delivery catheter 401 of the invention is highly trackable for ease of advancement through a vasculature.

During deployment of the embolic protection filter 405 in a vasculature, it is preferable to hold the mid section shaft 422 and proximal shaft 403 of the catheter body in a fixed position and move the distal portion 402 of the catheter body proximally over the mid section shaft 422 to facilitate deployment of the filter 405 from the reception space 404. In this manner, the location of the deployed filter 405 in the vasculature may be controlled for accurate filter deployment.

Referring now to FIGS. 44 and 45, there is illustrated another delivery catheter 501 according to the invention. In this case, the distal portion 502 of the catheter body has an extension sleeve 512 which extends proximally over at least part of the mid section shaft 522 of the catheter body, as illustrated in FIG. 44. The means for deploying a filter from within the reception space 504 is provided by an expandable chamber 511, the chamber 511 being defined between the mid section shaft 522 of the catheter body, the extension sleeve 512, a proximal seal 513, and a distal seal 514. The proximal seal 513 is fixed to the extension sleeve 512 and is slidably movable relative to the mid section shaft 522 of the catheter body in a sealed manner, and the distal seal 514 is fixed to the mid section shaft 522 of the catheter body and is slidably movable relative to the extension sleeve 512 in a sealed manner.

A fluid, such as compressed air, may be passed into the chamber 511 through one or more fluid ports 515 in a sidewall of the mid section shaft 522 of the catheter body. The internal fluid pressure in the chamber 511 pushes the proximal seal 513 proximally relative to the distal seal 514, and thereby moves the distal portion 502 of the catheter body proximally relative to the mid section shaft 522 of the catheter body to facilitate deployment of the medical device from within the reception space 504.

The mid section shaft 522 is constructed from a multi-lumen tube, in this case a dual lumen tube, so that it contains an inflation lumen 521 and a guidewire lumen 520. The mid section shaft 522 is connected to the proximal shaft 503 so that the inflation lumen 521 is continuous in the two shafts and is in communication with the fluid port 515.

In the delivery catheter501, the chamber 511 is expanded to facilitate filter deployment without the use of an inflatable balloon. This enables the catheter 501 to be constructed with small wall thicknesses for a particularly low crossing profile.

It is preferable that the seals 513, 514 are configured to prevent leakage of the expansion fluid from the chamber 511. In some cases, the expansion fluid may be chosen to be biocompatible to account for the possibility of some leakage from the chamber 511. A suitable biocompatible material is saline.

Deployment of a filter, using the delivery catheters of the invention only involve movement of a relatively short distal portion of the catheter body relative to the mid section shaft of the catheter body at the distal end of the catheter body. In this manner overall frictional losses in the delivery catheter system are minimised.

Due to the fluid expansion deployment action, only the expansion fluid is transmitted from the proximal end of the catheter to the distal portion of the catheter body. Tensile or compressive forces are not transmitted along the length of the catheter. The delivery catheters may thus be of relatively low tensile and compressive strengths. Accordingly the catheter materials may be chosen to ensure a particularly low profile, trackable delivery catheter.

In addition, the delivery catheters facilitate controlled deployment of a medical device.

Referring to FIGS. 46 to 48 there is illustrated another delivery catheter 570 according to the invention. The means for deploying a medical device 571 from the reception space 572 is directly engageable with the medical device 571 to facilitate movement of the medical device 571 distally relative to the distal portion 573 of the catheter body and thereby deploy the medical device 571 out of the reception space 572.

The deployment means comprises an engagement surface 574, which in this case is provided by a piston head, for pushing the medical device 571 distally relative to the distal portion 573 of the catheter body out of the reception space 572 to deploy the medical device 571. The piston head 574 is movable between a first configuration in which the piston head 574 is engaged with the medical device 571 which is within the reception space 572 (FIG. 46), for example during delivery of the medical device 571 to a desired site in a vasculature, and a second configuration in which the medical device 571 is pushed distally relative to the distal portion 573 of the catheter body out of the reception space 572 to deploy the medical device 571 (FIGS. 47 and 48).

The means for deploying the medical device 571 preferably comprises an expandable chamber 575 with a fluid port 576 through which a fluid, such as compressed air or saline solution, may be passed to expand the chamber 575 and move the piston head 574 between the first configuration and the second configuration. An expansion lumen 577 is provided in the catheter body which enables fluid to be passed from externally of the vasculature into the chamber 575 through the fluid port 576. The proximal wall 578 of the chamber 575 is fixed to the catheter body to ensure the fluid pressure within chamber 575 only moves the piston head 574 distally relative to the distal portion 573 of the catheter body.

FIG. 47 shows the medical device 571 partially deployed with the piston head 574 in between the first and second configuration. The piston head 574 contains a guidewire sleeve so that friction between the guidewire and the delivery catheter is minimised. FIG. 48 illustrates the medical device 571 fully deployed. The guidewire exit 579 is also shown indicating a rapid exchange construction.

This catheter allows for accurately controlled deployment of the medical device 571 as the piston head 574 moves proportionally to the volume of fluid which passes through the fluid port 576.

The invention is not limited to the embodiments hereinbefore described, with reference to the accompanying drawings, which may be varied in construction and detail.

1) A catheter comprising:
i. a catheter shaft;
ii. a pod defining a reception space for an embolic protection filter and an energy converter to convert energy into a translational movement of the filter relative to the pod;
iii. the filter being movable relative to the pod to facilitate deployment of a filter from within the reception space or retrieval of a filter into the reception space.

2) A catheter as claimed in claim 1 wherein the catheter shaft has a guidewire opening located a substantial distance distally of a proximal end of the catheter for rapid exchange of the catheter over a guidewire.

3) A catheter as claimed in claim 1 or 2 wherein the energy converter is configured to convert potential energy into a translational movement of the filter relative to the pod.

4) A catheter as claimed in any of claims 1 to 3 wherein the energy converter is configured to convert energy in the form of a pressurised fluid into a translational movement of the filter relative to the pod.

5) A catheter as claimed in any of claims 1 to 4 wherein the energy converter comprises an expandable chamber into which a pressurised fluid may be passed to move the filter relative to the pod:

6) A catheter as claimed in claim 5 wherein the expandable chamber is located substantially at a distal end of the catheter.

7) A catheter as claimed in claim 5 or 6 wherein the catheter comprises an expansion lumen in fluid communication with the expandable chamber for passing a pressurised fluid from a proximal end of the catheter to the expandable chamber.

8) A catheter as claimed in any of claims 5 to 7 wherein the expandable chamber has no relative moving parts.

9) A catheter as claimed in any of claims 5 to 7 wherein the expandable chamber is defined by an inflatable sac.

10) A catheter as claimed in claim 8 wherein the sac comprises an inner membrane and an outer membrane defining the chamber therebetween.

11) A catheter as claimed in claim 8 or 9 wherein the sac defines a central lumen through which a guidewire may pass.

12) A catheter as claimed in claim 10 when dependent on claim 9 wherein the central lumen is defined by the inner membrane.

13) A catheter as claimed in claim 8 or 9 wherein the sac is engageable with a guidewire for gripping a guidewire.

14) A catheter as claimed in claim 12 wherein the sac comprises an inner membrane, which, on expansion, grips the guidewire.

15) A catheter as claimed in any of claims 8 to 13 wherein the sac is of a non compliant material.

16) A catheter as claimed in any of claims 8 to 13 wherein the sac is of an elastomeric material.

17) A catheter as claimed in any of claims 8 to 15 wherein the sac has an abutment surface.

18) A catheter as claimed in claim 16 wherein the abutment surface is defined by a distal end of the sac.

19) A catheter as claimed in any of claims 1 to 17 wherein the energy converter is an inflation sac which is movable from a longitudinally retracted configuration to a longitudinally extended configuration.

20) A catheter as claimed in claim 18 wherein the sac is concertinered in the retracted configuration.

21) A catheter as claimed in claim 18 or 19 wherein the sac is of spiral form.

22) A catheter as claimed in any of claims 1 to 20 wherein a transition is provided between the catheter shaft and the pod.

23) A catheter as claimed in claim 21 wherein the transition comprises a mid section shaft.

24) A catheter as claimed in claim 21 or 22 wherein the transition defines a guidewire exit port.

25) A catheter as claimed in claim 23 wherein the exit port is a rapid exchange exit port.

26) A catheter as claimed in any of claims 21 to 24 wherein the transition tapers proximally from the pod towards the shaft.

27) A catheter as claimed in any of claims 21 to 25 wherein the longitudinal axis of the transition is offset from the axis of the catheter shaft.

28) A catheter as claimed in any of claims 21 to 26 wherein the catheter shaft extends through the transition for fluid communication with the expandable chamber.

29) A catheter as claimed in any of claims 1 to 27 wherein the catheter has a stop to control movement of the energy converter.

30) A catheter as claimed in claim 28 wherein the stop is a proximal stop to limit proximal movement of the energy converter.

31) A catheter as claimed in claim 28 or 29 wherein the stop is a distal stop to limit distal movement of the energy converter.

32) A catheter as claimed in any of claims 28 to 30 wherein the stop is provided on the catheter shaft and/or the pod.

33) A catheter as claimed in any of claims 1 to 31 wherein the catheter shaft has only a single lumen.

34) A catheter as claimed in claim 32 wherein the lumen is an inflation lumen. 35) A catheter as claimed in any of claims 1 to 33 wherein the catheter shaft is trackable.

36) A catheter as claimed in any preceding claim wherein the catheter shaft has a reinforcement.

37) A catheter as claimed in any of claims 1 to 35 wherein the pod extends co-axially around the catheter shaft along at least part of the catheter shaft.

38) A catheter as claimed in any of claims 5 to 36 wherein the expandable chamber is located between the pod and the catheter shaft.

39) A catheter as claimed in any of claims 5 to 37 wherein the expandable chamber is defined by an inflatable sac and an end of the sac is fixed to the shaft or the pod.

40) A catheter as claimed in claim 38 wherein one end of the sac is attached to the pod.

41) A catheter as claimed in claim 38 or 39 wherein one end of the sac is attached to the catheter shaft.

42) A catheter as claimed in any of claims 37 to 40 wherein the proximal end of the sac is attached to the pod and the distal end of the sac is attached to the catheter shaft.

43) A catheter as claimed in any of claims 1 to 41 wherein the catheter comprises an engagement element for engaging a filter in the reception space upon movement of the energy converter.

44) A catheter as claimed in claim 42 wherein the engagement element defines a guidewire lumen therethrough.

45) A catheter as claimed in claim 42 or 43 wherein the engagement element is attached to the catheter shaft.

46) A catheter as claimed in any of claims 42 to 44 wherein the engagement element extends distally of the catheter shaft.

47) A catheter as claimed in any of claims 42 to 45 wherein the engagement element is provided by a distal end face of the catheter shaft.

48) A catheter as claimed in any of claims 42 to 46 wherein the engagement element comprises an engagement surface for engaging a filter in the reception space.

49) A catheter as claimed in claim 47 wherein the engagement surface is provided by a distal end face of the engagement element.

50) A catheter as claimed in claim 47 or 48 wherein the engagement surface is configured to engage a tubular member of a filter.

51) A catheter as claimed in claim 49 wherein the tubular member defines a guidewire lumen therethrough.

52) A catheter as claimed in any of claims 1 to 50 wherein the catheter comprises a catheter shaft and a pod and the pod defines a guidewire exit port.

53) A catheter as claimed in claim 51 wherein the proximal end of the pod defines the exit port.

54) A catheter as claimed in claim 51 or 52 wherein a stop is provided in the pod.

56) A catheter as claimed in claim 53 wherein the stop is defined by the pod.

57) A catheter as claimed in any of claims 1 to 55 wherein a guidewire sleeve is provided in the pod.

58) A catheter as claimed in claim 56 wherein the guidewire sleeve defines a distal abutment.

59) A catheter as claimed in any of claims 1 to 57 wherein the pod is of an expansile material.

60) A catheter as claimed in any of claims 1 to 58 wherein the inflation lumen for the sac is substantially concentric with the guidewire lumen over at least a portion of the length thereof.

61) A catheter as claimed in any of claims 1 to 59 wherein the sac is of a coil form.

62) A catheter as claimed in claim 60 wherein the coil is of a spiral form.

63) A catheter as claimed in any of claims 1 to 61 having a wire gripper.

64) A catheter as claimed in claim 62 wherein the wire gripper is provided by an expandable sac which, in an expanded configuration, is used to grip a guidewire.

65) A catheter as claimed in any of claims 1 to 63 wherein the pod at least partially provides the energy converter.

66) A catheter as claimed in any of claims 1 to 64 wherein the pod is longitudinally movable relative to the catheter shaft.

67) A catheter as claimed in claim 65 wherein the pod is movable from a retracted configuration to an extended configuration for deployment of a filter from the pod or for retrieval of a filter into the pod.

68) A catheter as claimed in any of claims 64 to 66 wherein the pod is tethered.

69) A catheter as claimed in claim 67 wherein a tether extends from the pod to a proximal handle end of the catheter.

70) A catheter as claimed in claim 68 wherein the tether is a tether wire.

71) A catheter as claimed in any preceding claim wherein the catheter is a delivery catheter and the pod defines a reception space for delivery of a filter to a desired site.

72) A catheter as claimed in any of claims 1 to 69 wherein the catheter is a filter retrieval catheter and the pod defines a reception space for a retrieved filter.

73) A method of delivering a filter comprising the steps of:
i. providing a delivery catheter comprising a catheter shaft, an energy converter and a reception space;
ii. placing a filter in the reception space;
iii. advancing the delivery catheter and filter through the vasculature to a target deployment site;
iv. deploying the filter at the target site with the assistance of fluid pressure.

74) The method of claim 73 wherein the method further comprises the step of removing the delivery catheter.

75) The method of claim 73 wherein the force of delivery is delivered entirely by fluid pressure.

76) The method of claim 73 wherein the delivery catheter and filter are advanced through the vasculature over a guidewire.

77) The method of claim 73 wherein the delivery catheter and filter are advanced through the vasculature on a guidewire.

78) A method of retrieving a filter comprising the steps of:
i. providing a retrieval catheter comprising a catheter shaft, an energy converter and a reception space;
ii. advancing the retrieval catheter through the vasculature to the retrieval site;
iii. retrieving the filter at the target site with the assistance of fluid pressure.

79) The method of claim 78 wherein the method further comprises the step of removing the retrieval catheter and filter.

80) The method of claim 78 wherein the force of retrieval is delivered entirely by fluid pressure.

81) The method of claim 78 wherein the retrieval catheter is advanced through the vasculature over a guidewire.

82) A method of deploying or retrieving a filter comprising the steps of:
i. displacing a volume of fluid at a proximal end of a catheter;
ii. converting the displaced volume of fluid into a displacement at the distal end of a catheter;
iii. the displacement effecting a deployment or retrieval of a filter.

83) A catheter substantially as hereinbefore described with reference to the accompanying drawings.
